(19) United States
(12) Patent Application Publication
(10) Pub. No.: US 2004/0116733 A1
(43) Pub. Date: Jun. 17, 2004

(54) Composition and compound based on salt (s) of metals and of acid exhibiting a sulfonyl group carried by a perhalogenated carbon and their use as lewis acid

(76) Inventors: Jacques Dubac, Pechbusque (FR); Christophe Le Roux, Chateauroux (FR); Sigrid Repichet, Louviers (FR); Nicolas Roques, Lyon (FR); Jean-Marie Bernard, Mornant (FR); Jean-Pierre Maestro, Saint-Symphorien D'Ozon (FR); Thierry Vidal, Lyon (FR); Magali Peyronneau, Toulouse (FR); Alexandre Picot, Saint Plancard (FR); Stephane Mazieres, Castanet Tolosan (FR)

Correspondence Address: BURNS DOANE SWECKER & MATHIS L L P, POST OFFICE BOX 1404, ALEXANDRIA, VA, 22313-1404, US

(21) Appl. No.: 09/903,635

(22) Filed: Jul. 13, 2001

Related U.S. Application Data
(60) Provisional application No. 60/217,923, filed on Jul. 13, 2000.

(30) Foreign Application Priority Data
Dec. 29, 2000 (FR) 00/17310

(51) Int. Cl. C07C 309/00

(52) U.S. Cl. 562/78

(57) ABSTRACT

The present invention relates to a bismuth-type promoter and its use as a Lewis acid catalyst for acylation reactions of aromatic compounds. The promoter corresponds to the formula MY_{μ-q}ξ_{q}, where M represents a μ-valent and at least trivalent element in the cationic form, preferably known to give Lewis acids, where Y is a monovalent anion or a monovalent anionic functional group, where ξ⁻ represents an anion or an anionic functional group carrying a sulfonyl functional group carried by a perhalogenated atom, preferably a perfluorinated atom, more preferably a perfluoromethylene (—CF₂—); and where q is an integer advantageously chosen within the closed range (comprising the limits) ranging from 1 to (μ−1) (that is to say, 1 or 2 when μ is 3). The present application also relates to processes for the preparation of the promoter.

A subject matter of the present invention is a novel category of catalyst reacting as a Lewis acid.

There already exist catalysts based on Lewis acid which are used to carry out numerous reactions and in particular to carry out reactions referred to as Friedel-Crafts reactions or reactions for the alkylation of aromatic nuclei. In general, these catalysts promote the formation of cations and in particular of carbocations.

The most commonly used catalysts are trivalent atoms, generally metallic in nature, which exhibit an electron vacancy capable of capturing leaving groups, which then constitute anions or entities which it is conventional to write in the form of anions. Thus, the best known of these catalysts, aluminum trichloride, is capable of detaching a chlorine from an acyl chloride and of forming the corresponding carbocation; this carbocation will then act as electrophile, which will make it possible to give rise to numerous reactions from an esterification reaction, to give an ester, to the acylation of an aromatic nucleus.

It should be pointed out, at this stage in the introduction, that one of the most difficult reactions to carry out is the sulfonylation reaction, in particular the alkanesulfonylation reaction. In general, the latter reactions are not possible with acid chlorides but only with acid anhydrides where two sulfonyl radicals are bonded by an oxygen. To date, apart from the sulfonates which form the subject matter of the present invention, only boron tristriflate, but in a stoichiometric amount, had made the reaction possible from alkanesulfonyl halides. It is this reaction which is used as main test in the present application.

There already exist numerous Lewis acids known to a person skilled in the art, but the field is still on the lookout for highly active catalysts which either will act at a very low dose or will act on products which are difficult to ionize.

Numerous studies have recently been carried out using salts of triflic acid and of various metals as catalysts.

These triflic acid salts have proved to be powerful catalysts, indeed even excessively powerful catalysts.

However, these salts are extremely expensive, triflic acid and the triflic anion being very difficult to obtain at prices sufficiently low to render the use of these catalysts exploitable on an industrial scale.

This is why one of the aims of the present invention is provide a novel family of catalysts which exhibit similar properties to the salts of triflic acid without having the cost thereof. This is because trivalent cations require the presence of three triflate anions in order to provide for the electrical balance of the molecule or of the salt. Mutatis mutandis, the problem is the same for polyvalent cations, in particular tetra- and pentavalent cations, and for imides.

Another aim of the present invention is to find a process which makes it possible to use these novel catalysts.

Another aim of the present invention is to provide a process which makes it possible to easily achieve the catalysts according to the present invention.

Another aim of the present invention is to provide novel compounds capable of acting as catalysts according to the present invention.

These aims and others which will appear subsequently are achieved by means of the use as catalysts of salt of element referred to as M, the valency of which is greater than or equal to 3, advantageously equal to 3, comprising, as coanions, at least one and at most (μ−1) (that is to say, at most two when, that is to say, when the element is trivalent) anions carrying a sulfonyl functional group carried by a perhalogenated atom (that is to say, directly connected to said perhalogenated atom), preferably a perfluorinated atom, more preferably a perfluoromethylene (—CF₂) group.

μ is advantageously at most equal to five, preferably to 4.

In the present description, the halogens, in particular chlorine and fluorine, are considered to be perhalogenated, so that the chloro- and fluorosulfonic anions are targeted by the definition of the sulfonate ions above. However, in particular in the case where use is made of reaction mixtures comprising water, these sulfonates can hydrolyze; consequently, it is usually preferable to use them perhalogenated on the carbon.

The cations targeted by the present invention are essentially those of the rare earth metals (scandium, yttrium, lanthanum and lanthanide) and metals of the square in the Periodic Table formed by gallium, germanium, arsenic, indium, tin, antimony, thallium, lead and bismuth. This is particularly the case if they have a valency of greater than or equal to 3.

The catalysts according to the present invention give good results even if they are hydrated, this being the case up to levels of hydration ranging up to 12 H₂O (per element M). The limit is related more to the hydrolyzable nature (stricto sensu) of the substrates than to the sensitivity to water of the compounds according to the present invention. However, it should be noted that the strength of the Lewis acid according to the invention generally decreases with hydration. It is thus preferable to limit the hydration to 3 H₂O; a hydration ranging from ½ to 3 H₂O per atom of element M generally constitutes a good compromise for nonaqueous and/or anhydrous media.

The other anion or the other anions are organic or inorganic anions, preferably monoanions.

Mention may be made, among these anions, referred to as Y⁻, of sulfonates, monoalkyl sulfates (when the latter are stable in the medium), carboxylates, halides, halogenates (when the latter are not too oxidizing for the medium), or phosphates, phosphonates and phosphinates; pyrophosphates can be envisaged in media where they are stable; carbonates and bicarbonates; O⁻ functional groups, as in oxides (O⁻), indeed even hydroxides, can give highly active compounds. When the ξ⁻ anions, that is to say the anions carrying a sulfonyl functional group carried by a perhalogenated atom, are sulfonates, aromatic carbanions are, however, to be avoided as the loss in activity is significant. On the other hand, in the case where the charge is carried by a nitrogen and in particular in the case of imides, the loss in activity is low.

Thus, the preferred compounds according to the present invention correspond to the formula:

MY_{μ-q}ξ_{q}

where M represents a μ-valent and at least trivalent element in the cationic form, preferably known to give Lewis acids;

where Y is a monovalent anion or a monovalent anionic functional group;

where ξ⁻ represents an anion or an anionic functional group carrying a sulfonyl functional group carried by a perhalogenated atom, preferably a perfluorinated atom, more preferably a perfluoromethylene (—CF₂—) group; and

where q is an integer advantageously chosen within the closed range (comprising the limits) ranging from 1 to (μ−1) (that is to say, 1 or 2 when μ is 3).

The compounds according to the present invention can be used alone or as a mixture and in particular as a mixture with one another. They can be as a mixture with the starting material and with the compound of formula Mξ_{μ}, which would correspond to complete electrical neutrality given by the ξ⁻ alone.

For this reason, the compositions used can have fractional values. Thus, if M^{μ+} is the at least trivalent cation and if Y⁻ denotes the anions other than ξ⁻, the compounds according to the present invention correspond to the formula MY_{μ-q}ξ_{q}, with q equal to 1, 2, μ−2 and/or μ−1. In the case where the catalytic compositions comprise mixtures, q can become fractional and, in particular, can be between 0.1 and μ−0.1 (that is to say, between 0.1 and 2.9 when M is trivalent), advantageously from 0.5 to μ−0.5 (0.5 to 2.5 when M is trivalent), preferably from 1 to μ−1 (from 1 to 2 when M is trivalent), inclusive. When the use is envisaged of ξ⁻ anions comprising two or more (identical or different) ξ⁻ functional groups carried by a perhalogenated carbon atom, this polyfunctionality has to be taken into account in the value of q. In that case, q would involve the number of equivalents of ξ⁻ functionality.

In general, it is preferable for the anions other than the perhalogenated sulfonates not to be chelating. It is also preferable, in general, for the Kₐ of the acid associated with these anions to be at most equal to approximately 10, preferably to approximately 5, more preferably to approximately 2.

It is preferable for the acid associated with these anions not to be more acidic than hydrohalic acids.

It is also preferable for these anions not to be complex (that is to say, resulting from the complexing of a cation with simple anions in an amount which is sufficient to produce an anionic complex) or excessively bulky anions (such as BF₄⁻, PF₆⁻, and the like, as these anions are both bulky and capable of dissociating).

The cations of these novel catalysts are, as has been mentioned, cations which are advantageously trivalent in nature and are preferably chosen from the periods of the Periodic Table at least equal to the third, preferably to the fourth.

Mention may be made, as cation of particular interest, of those already mentioned, namely gallium, germanium, arsenic, indium, tin, antimony, thallium, lead and bismuth; those of most interest are those in the trivalent state and the preferred state is the trivalent state. Thus, according to the present invention, gallium(III), indium(III), antimony(III) and even arsenic(III) are preferred, as well as bismuth and the rare earth metals, including scandium and yttrium.

The anions carrying a sulfonyl functional group carried by a perhalogenated atom are the anions in which the charge is carried by the atom directly bonded to the sulfonyl functional group. Thus, the sulfone group (SO₂) of the sulfonyl is bonded, on the one hand, to the perhalogenated atom and, on the other hand, to the atom carrying the anionic charge or hydrogen, when the anion is in the form of its associated acid.

The preferred anions are those which correspond to the general formula (I):

R₁—Z⁻—SO₂—R_{f}

where Z represents an atom from the nitrogen column or a chalcogen;

where, when Z represents an atom from the nitrogen column, R₁ represents an electron-withdrawing radical, advantageously chosen from those of formula (II):

*[SO₂]ᵥR_{f}′

where v here is zero or 1, advantageously 1

where R_{f}′ and R_{f} independently represents a fluorine, a carbonaceous radical in which the carbon connected to the sulfur is perfluorinated, or a halogen atom heavier than fluorine;

with the condition that, when Z represents a chalcogen, R₁ does not exist.

Z is advantageously nitrogen or oxygen.

Another preferred value of R₁ can be an arylsulfonyl group (such as benzenesulfonyl) or an aliphatic sulfonyl group (such as alkanesulfonic, for example the mesylate).

The total carbon number of these anions is advantageously at most 15, preferably at most 10.

In fact, according to one aspect of the present invention, and taking, for teaching purposes, the case of perfluoroalkanesulfonic acids, for example, it has been shown that the mixed salts of trivalent cations and of sulfonic acids carried by a perfluorinated carbon have catalytic properties similar to pure triflates, that is to say triflates for which the sole anion providing electrical neutrality is that resulting from triflic acid (i.e. trifluoromethanesulfonic).

This invention is of essential economic advantage as, on the one hand, it is extremely difficult to obtain these triflates pure, the exchange with conventional salts being very difficult to bring to completion; this is because it is fairly common to use acetates for preparing triflates by displacing the acetic anion with the triflic anion, the first acetic is generally fairly easy to displace, the second is already more difficult, as for the third, the techniques become extremely problematic, in particular if it is desired to obtain an anhydrous salt. Examples of a difficulty in preparing these salts are provided in the patent application filed on behalf of the Applicant Company published under No. EPA 0 877 726.

Furthermore, triflic acid and the triflates which result therefrom are particularly expensive. The fact of having shown that it is possible to have cases of catalytic properties with conventional anions neutralizing the trivalent cation with the triflates is of very great advantage.

It is advisable, among the anions corresponding to acids perfluorinated on the carbon carrying the sulfonic functional group, to mention the anions corresponding to the following general formula:

R_{f}—SO₂—O⁻

or:

R_{f}—SO₂—N(R₁)—

with R_{f} and/or R_{f}′, which are identical or different, denoting:

EWG—(CX₂)ₚ—

[lacuna]

the X groups, which are alike or different, represent a fluorine or a radical of formula CₙF₂ₙ₊₁, with n an integer at most equal to 5, preferably to 2;

p represents zero or an integer at most equal to 2, with the proviso that, when p represents zero, EWG is chlorine and especially fluorine;

EWG represents a hydrocarbonaceous group, that is to say carrying hydrogen and carbon, such as alkyl or aryl, preferably having at most 15 carbon atoms, or instead an electron-withdrawing group (that is to say, the Hammet constant σₚ of which is greater than 0, advantageously than 0.1, preferably than 0.2), the possible functional groups of which are inert under the reaction conditions, advantageously fluorine or a perfluorinated residue of formula CₙF₂ₙ₊₁, with n an integer at most equal to 8, advantageously to 5.

The greater the value of p, the greater the solubility of the salts in organic solvents which are not very miscible with water (solubility of said solvents of less than or equal to 1% by mass); consequently, it is preferable for p to be at least equal to 1, and even to 2, when it is desired to operate in media which are not very miscible with water.

The total number of carbons of R_{f} is advantageously between 1 and 15, preferably between 1 and 10.

EWG can be or can carry a sulfonyl functional group, including a sulfonic acid of the type of that described above or its anion.

EWG can also constitute a bond with a polymeric network, although this is not preferred.

A special mention must be made of sulfonic acids comprising two sulfonic functional groups, themselves both carried by a perhalo group, preferably perfluoroethylene or perfluoromethylene.

The distance between two sulfonic functional groups is then advantageously, by the shortest route, less than 10, preferably than 5, more preferably than 4 chain units.

The compounds according to the present invention can be used alone or as a mixture and in particular as a mixture with one another. They can be as a mixture with the starting material and with the sulfonate which would correspond to complete electrical neutrality given by the sulfonates according to the present invention.

For this reason, the compositions used can have fractional values. Thus, if M^{μ+} is the at least trivalent cation, if Rₓ—SO₂—O⁻ denotes the perhalogenated sulfonate and if Y⁻ denotes the anions other than the sulfonates carried by a perhalogenated carbon, the compounds according to the present invention correspond to the formula

MY_{μ-q}(RₓSO₂—O⁻)_{q},

with q equal to 1, 2, μ−2 and/or μ−1. In the case of catalytic compositions which comprise mixtures, q can become fractional and in particular can be between 0.1 and μ−0.1, (that is to say, between 0.1 and 2.9 when M is trivalent), advantageously from 0.5 to μ−0.5 (0.5 to 2.5 when M is trivalent), preferably from 1 to μ−1 (from 1 to 2 when M is trivalent), inclusive. When the use is envisaged of sulfonates comprising two or more sulfonate functional groups carried by a perhalogenated carbon atom, it is necessary to take into account this polyfunctionality in the value of q. In that case, q will involve the number of equivalents of sulfonate functionality carried by perhalogenated carbon atoms.

These compounds can be used as Lewis acids, as was mentioned above, and in reactions where Lewis acids are used as catalysts. They can in particular be used to functionalize aromatic nuclei by reactions employing nucleophilic cations. In particular, it is possible to carry out reactions with acid halides or acid anhydrides which give a cation such as the sulfonylium cation or the acylium cation.

Although this reaction does not generally require powerful catalysts, this reactant can also be used for alkylation reactions.

Generally, these catalysts constitute Lewis acids which are particularly suitable for forming nucleophilic cations in particular from acid anhydrides, whether symmetrical or asymmetrical.

It may be considered that acid chlorides are a form of asymmetric acid anhydride, one of the acids being a hydrohalic acid. In particular, these acid chlorides, in the case of sulfonylations, although they are supposed to be less active than symmetrical anhydrides, give excellent yields when they are used concomitantly with the catalysts according to the invention.

It should also be noted that these compounds are capable of being very good catalysts of reaction in a neutral medium, such as, for example, aldolization or ketolization reactions.

These catalysts can be made in situ in the case of rare earth metals (scandium, yttrium, lanthanum and lanthanide) and elements from the square of the Periodic Table formed by gallium, germanium, arsenic, indium, tin, antimony, thallium and lead. The case of bismuth is more complex, due in particular to the difficulty in synthesizing bismuth trifluoromethylsulfonates by simple action of triflic acid (TfOH).

Thus, for the cations targeted above by the in situ route, it will not be departing from the invention to add an acid ξH, such as perhalogenated sulfonic acids (see above), to a salt of the above elements, namely rare earth metals (scandium, yttrium, lanthanum and lanthanide), gallium, germanium, arsenic, indium, tin, antimony, thallium and lead, in particular if the amount of acid (for example triflic or sulfonimide) is less than that necessary for the complete replacement of the anions (including the oxide [O⁻] and hydroxide anions) providing the initial neutrality of said salt; when they are not oxides or hydroxides, it is preferable to displace a portion, advantageously at least 1/(2μ), preferably at least 1/μ, of the initial anions, generally by distillation, when this is possible. The displacement of oxygen-comprising anions, oxide, hydroxide or carbonate, leaves water of formation in the medium which does not detrimentally affect the catalysis to a significant extent. Of course, divalent anions count for two.

Thus, according to the present invention, it is possible to use, as catalyst of Lewis acid type, a composition comprising at least one of the salts chosen from the group of the salts of rare earth metals (scandium, yttrium, lanthanum and lanthanide), of gallium, of germanium, of arsenic, of indium, of tin, of antimony, of thallium and of lead and of an acid ξH (such as sulfonics, that is to say sulfonic acids in which the sulfonic acid group is carried by a perhalogenated atom above, sulfonimides in which a sulfonyl functional group is carried by a perhalogenated atom, and, if appropriate, their mixture, but the mixtures are not preferred); that is to say, acids comprising sulfonyl group(s) carried by a perhalogenated atom, preferably a perfluorinated atom, more preferably a perf luoromethylene (—CF₂) group. As is mentioned in the present application, such a composition can comprise, inter alia, solvents and water when the agent generating the cation is not sensitive to hydrolysis under the operating conditions.

Thus, the present invention provides a reactant of use in aromatic electrophilic substitutions (such as Friedel-Crafts reactions) which comprises:

at least one salt chosen from the at least trivalent salts of the elements chosen from rare earth metals (scandium, yttrium, lanthanum and lanthanide), gallium, germanium, arsenic, indium, tin, antimony, thallium and lead;

at least one acid ξH such as sulfonics, that is to say sulfonic acids in which the sulfonic functional group is carried by a perhalogenated atom above, sulfonimides in which a sulfonyl group is carried by a perhalogenated atom, and, if appropriate, their mixture, but the mixtures are not preferred); that is to say, acids comprising sulfonyl group(s) carried by a perhalogenated atom, preferably a perfluorinated atom, more preferably a perfluoromethylene (—CF₂) group;

a substituting agent capable of giving an electrophilic cation and advantageously chosen from acid anhydrides and more particularly acid halides;

the ratio in equivalents of said ξH functional groups, such as sulfonics, to said element being at least equal to 0.05, advantageously to 0.1, preferably to 0.5.

Said ratio is advantageously at most equal to μ−0.1, preferably to μ−0.5, more preferably to μ−1.

Said composition can additionally comprise a solvent, which can moreover be a possible substrate in excess.

As regards the substituting agents, the acids can be polyacids and halides, polyacid polyhalides and in particular the monohalide and dihalide of sulfur-based acids.

By choosing the operating conditions, in particular temperature, it is then possible to carry out one or more condensations on the polyhalide.

The effectiveness of the catalysts according to the present invention makes it possible to choose operating conditions which allow the final unstable compounds to survive. Thus, it has been shown that BiCl(OTf)₂ was already active with thionyl chloride at a temperature of −5° C., thus making possible the 99% synthesis of arylsulfinyl chloride (ArSOCl), which ordinarily are not stable at high temperatures.

According to a preferred alternative form of the present invention, the salts according to the present invention correspond to the formula (that is to say that, in the preceding formula, Z is oxygen and consequently R₁ does not exist)

MY_{μ-q}(RₓSO₂—O⁻)_{q},

where M is an element in an at least trivalent cationic form, which element being advantageously chosen from the rare earth metals (scandium, yttrium, lanthanum and lanthanide) and the metals of the square of the Periodic Table formed by gallium, germanium, arsenic, indium, tin, antimony, thallium, lead and bismuth;

where μ represents the charge of the cation corresponding to M;

where Y represents the anions, other than the sulfonates perhalogenated on the carbon carrying said sulfonate functional group;

where q represents an integer chosen within the closed range from 1 to μ−1; q can in particular take the values 1, 2, μ−2 and/or μ−1;

and can be made in situ (except for bismuth) or prepared in isolation.

These salts make possible in particular catalyses in media where an excessively high acidity can be harmful (acidity corresponding to an acidity which, if the salt were in aqueous medium, would correspond to a pH of between 2 and 8, advantageously between 4 and 7). The salts in themselves are not acidic and can be used in neutral medium (acidity corresponding to an acidity which, if the salt were in aqueous medium, would correspond to a pH of between 2 and 8, advantageously between 4 and 7). This state of affairs makes it possible to use in a neutral medium catalyst of Lewis acid type which is both powerful and which does not significantly modify the neutrality of the medium.

According to another alternative form of the present invention, the salts according to the present invention correspond to the formula (that is to say that, in the preceding formula, Z is nitrogen):

MY_{μ-q}(RₓSO₂—N(R₁)⁻)_{q},

with Rₓ having the value of R_{f} and R₁ being an electron-withdrawing group, advantageously an aromatic or aliphatic sulfonyl radical and preferably a sulfonyl radical carried by a perhalogenated atom as defined in the beginning of the present description;

where M is an element in an at least trivalent cationic form, which element being advantageously chosen from the rare earth metals (scandium, yttrium, lanthanum and lanthanide) and the metals of the square of the Periodic Table formed by gallium, germanium, arsenic, indium, tin, antimony, thallium, lead and bismuth;

where μ represents the charge of the cation corresponding to M;

where Y represents the anions, other than the sulfonates perhalogenated on the carbon carrying said sulfonate functional group;

where q represents an integer chosen within the closed range from 1 to μ−1; q can in particular take the values 1, 2, μ−2 and/or μ−1;

and can be made in situ or prepared in isolation.

These salts make possible in particular catalyses in media where an excessively high acidity can be harmful (acidity corresponding to an acidity which, if the salt were in aqueous medium, would correspond to a pH of between 2 and 8, advantageously between 4 and 7). The salts in themselves are not acidic and can be used in neutral medium (acidity corresponding to an acidity which, if the salt were in aqueous medium, would correspond to a pH of between 2 and 8, advantageously between 4 and 7). This state of affairs makes it possible to use in a neutral medium catalyst of Lewis acid type which is both powerful and which does not significantly modify the neutrality of the medium.

It should be noted that, when Y is Cl and M is Bi, whatever the amount of the imide (such as tfsi), it is impossible to prepare the trisimide in situ; only the monoimide is easily prepared.

These imide anions, specific cases of ξ⁻, advantageously correspond to the formula (II):

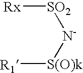

in which:

Rₓ has the value defined previously and advantageously represents a fluorine atom or advantageously an organic carbonaceous radical, if appropriate substituted by one or more halogen atoms, the carbon of which carrying the sulfonic functional group is perhalogenated, preferably perfluorinated, with Rₓ and R₁′ being able to be bonded to one another,

k is equal to 1 or 2, with k preferably being equal to 2 when R₁′ represents a fluorine atom,

R₁′ is an organic carbonaceous radical advantageously comprising at most 30 carbon atoms [when it is not polymeric (that is to say, does not constitute a bond for joining to a polymer)] or a group as defined for Rₓ, and the value k advantageously being 2.

In the case where M is bismuth, the formula of the salts which are targeted by the invention can be:

(R₃)_{(μ-q)}Biξ_{q}

with:

μ equal to three;

ξ corresponding to the formula II;

q representing the integer 1 or 2; and

the R₃ group(s), which are identical or different, chosen from

the Y⁻ anions, advantageously a carboxylate group, such as acetate or sulfate, or a halogen atom, preferably chlorine, bromine and iodine;

the phenyl groups, if appropriate substituted by one or more electron-donating substituents of linear or branched C₁ to C₄ alkyl type, such as, for example, methyl, ethyl or propyl, of C₁ to C₄ alkoxy type, such as methoxy, ethoxy, propoxy or phenoxy, or of C₁ to C₄ thioether type.

Preferably, when q is equal to 1, the two R₃ groups are identical.

According to a preferred alternative form of the invention, the anion of formula (I) corresponds to the formula (IIa) or (IIb):

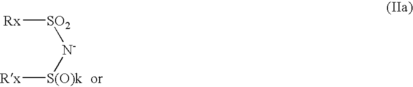

or

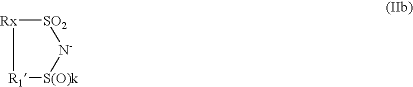

with, in the case of the formula (Ib), Rₓ and Rₓ′ having to represent a hydrocarbonaceous chain in agreement with the definitions provided above for Rₓ.

As regards the anion of formula (I), it corresponds in particular to the formula:

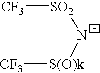

with k representing 1 or 2, and preferably 2.

According to a preferred embodiment of the invention, x has the value 1.

As regards the combination between bismuth and the two types of anions, it can be anionic or nonionic in nature.

The compounds as defined above prove to be particularly effective as Lewis acids. This thus results in an increased catalytic activity of said promoter.

Mention may more particularly be made, by way of representation of the promoters claimed according to the invention, of BiPh(NTf₂)₂ and BiPh₂(NTf₂).

The salts of elements of valency μ which are targeted by the present invention generally exhibit particularly advantageous Lewis acid properties.

The catalyst promoters claimed have thus proved to be particularly effective in catalyzing reactions of the following types: Diels-Alder reactions, carbonyl allylations, ene reactions and Prins reactions.

In addition, specific mention may be made of reactions where a carbonyl is activated by a Lewis acid and is added to an unsaturation, generally an activated unsaturation, such as enol or enol ether (see aldolization example). It is advantageous to note that, for this type of reaction, the mixed salt is suitable for the aqueous medium.

Mention may also be made of the openings and polycondensations of cyclic ethers, including epoxides. In the latter case, it is advisable to be positioned in the lower part of the range of hydrations.

Mention may also be made of the openings and polycondensations of cyclic esters (lactones).

More particularly, another subject matter of the present invention is the use of a promoter comprising at least one anion of formula (I) as defined above and one cation of formula (III).

In order to give a better explanation of the scope of the invention, it may in particular be indicated that it is possible, by using the catalysts according to the present invention, to carry out a sulfonylation or an acylation of aromatic compounds corresponding to the general formula (1)

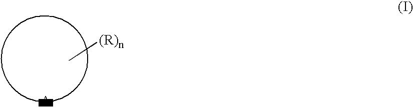

in which:

A symbolizes the residue of a ring forming all or part of a monocyclic or polycyclic, aromatic, carbocyclic or heterocyclic system, it being possible for the said cyclic residue to carry a radical R representing a hydrogen atom or one or more identical or different substituents,

n represents the number of substituents on the ring.

The invention applies in particular to the aromatic compounds corresponding to the formula (I) in which A is the residue of a cyclic compound preferably having at least 4 atoms in the optionally substituted ring and representing at least one of the following rings:

a monocyclic or polycyclic aromatic carbocycle,

a monocyclic or polycyclic aromatic heterocycle comprising at least one of the heteroatoms O, N and S.

To be more specific, without for all that limiting the scope of the invention, the optionally substituted residue A represents the residue:

1) of a monocyclic or polycyclic aromatic carbocyclic compound.

The term “polycyclic carbocyclic compound” is understood to mean:

a compound composed of at least 2 aromatic carbocycles which form, with one another, ortho- or ortho- and peri-condensed systems,

a compound composed of at least 2 carbocycles, of which only one among them is aromatic, which rings form, with one another, ortho- or ortho- and peri-condensed systems.

2) of a monocyclic or polycyclic aromatic heterocyclic compound.

The term “polycyclic heterocyclic compound” defines:

a compound composed of at least 2 heterocycles comprising at least one heteroatom in each ring, at least one of the two rings of which is aromatic, which rings form, with one another, ortho- or ortho- and peri-condensed systems,

a compound composed of at least one hydrocarbonaceous ring and at least one heterocycle, at least one of the rings of which is aromatic, which rings form, with one another, ortho- or ortho- and peri-condensed systems.

3) of a compound composed of a sequence of rings as defined in paragraphs 1 and/or 2 bonded to one another:

via a valency bond,

via an alkylene or alkylidene radical having from 1 to 4 carbon atoms, preferably a methylene or isopropylidene radical,

via one of the following groups:

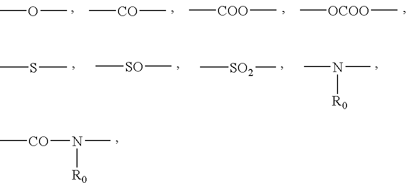

in these formulae, R₀ represents a hydrogen atom, an alkyl radical having from 1 to 4 carbon atoms, a cyclohexyl radical or a phenyl radical.

Mention may be made, as examples of rings under 1) to 3), of:

1) benzene, toluene, xylene, naphthalene or anthracene,

2) furan, pyrrole, thiofene, isoxazole, furazan, isothiazole, imidazole, pyrazole, pyridine, pyridazine, pyrimidine, quinoline, naphthyridine, benzofuran or indole,

3) biphenyl, 1,1′-methylenebiphenyl, 1,1′-isopropylidenebiphenyl, 1,1′-oxybiphenyl or 1,1′-iminobiphenyl.

In the process of the invention, use is preferably made of an aromatic compound of formula (I) in which A represents a benzene nucleus.

The aromatic compound of formula (I) can carry one or more substituents.

The number of substituents present on the ring depends on the carbon condensation of the ring and on the presence or absence of unsaturations in the ring.

The maximum number of substituents which can be carried by a ring is easily determined by a person skilled in the art.

In the present text, the term “more” is understood to mean generally less than 4 substituents on an aromatic nucleus. Examples of substituents are given below but this list does not have a limiting nature. As mentioned above, the substituents may or may not activate the aromatic nucleus.

The residue A can optionally carry one or more substituents which are represented in the formula (I) by the symbol R and the preferred meanings of which are defined below:

the R radical or radicals represent one of the following groups:

a hydrogen atom,

a linear or branched alkyl radical having from 1 to 6 carbon atoms, preferably from 1 to 4 carbon atoms, such as methyl, ethyl, propyl, isopropyl, butyl, isobutyl, sec-butyl or tert-butyl,

a linear or branched alkenyl radical having from 2 to 6 carbon atoms, preferably from 2 to 4 carbon atoms, such as vinyl or allyl,

a linear or branched alkoxy radical having from 1 to 6 carbon atoms, preferably from 1 to 4 carbon atoms, such as the methoxy, ethoxy, propoxy, isopropoxy or butoxy radicals,

a cyclohexyl radical,

an acyl group having from 2 to 6 carbon atoms,

a radical of formula:

—R₁—OH

—R₁—COOR₂

—R₁—CHO

—R₁—NO₂

—R₁—CN

—R₁—N(R₂)₂

—R₁—CO—N(R₂)₂

—R₁—X

—R₁—CF₃

in the said formulae, R₁ represents a valency bond or a saturated or unsaturated, linear or branched, divalent hydrocarbonaceous radical having from 1 to 6 carbon atoms, such as, for example, methylene, ethylene, propylene, isopropylene or isopropylidene; the radicals R₂, which are identical or different, represent a hydrogen atom or a linear or branched alkyl radical having from 1 to 6 carbon atoms; X symbolizes a halogen atom, preferably a chlorine, bromine or fluorine atom.

When n is greater than or equal to 2, two R radicals and the 2 successive atoms of the aromatic ring can be bonded to one another via an alkylene, alkenylene or alkenylidene radical having from 2 to 4 carbon atoms to form a saturated, unsaturated or aromatic heterocycle having from 5 to 7 carbon atoms. One or more carbon atoms can be replaced by another heteroatom, preferably oxygen. Thus, the R radicals can represent a methylenedioxy or ethylenedioxy radical.

The present invention applies very particularly to the aromatic compounds corresponding to the formula (I) in which:

the R radical or radicals represent one of the following groups:

a hydrogen atom,

an OH group,

a linear or branched alkyl radical having from 1 to 6 carbon atoms,

a linear or branched alkenyl radical having from 2 to 6 carbon atoms,

a linear or branched alkoxy radical having from 1 to 6 carbon atoms,

a —CHO group,

an acyl group having from 2 to 6 carbon atoms,

a —COOR₂ group, where R₂ has the meaning given above,

an —NO₂ group,

an —NH₂ group,

a halogen atom, preferably fluorine, chlorine or bromine,

a —CF₃ group,

n is a number equal to 0, 1, 2 or 3.

Use is more particularly made, among the compounds of formula (I), of those corresponding to the following formulae:

a monocyclic or polycyclic aromatic carbocyclic compound with rings which can form, with one another, an ortho-condensed system corresponding to the formula (Ia):

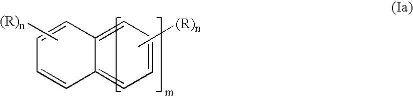

in the said formula (Ia), m represents a number equal to 0, 1 or 2 and the symbols R, which are identical or different, and n having the meanings given above,

a compound composed of a sequence of two or more monocyclic aromatic carbocycles corresponding to the formula (Ib):

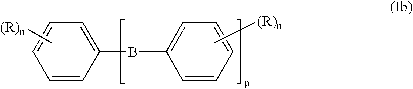

in the said formula (Ib), the symbols R, which are identical or different, and n have the meaning given above, p is a number equal to 0, 1, 2 or 3 and B represents:

a valency bond,

an alkylene or alkylidene radical having from 1 to 4 carbon atoms, preferably a methylene or isopropylidene radical,

one of the following groups:

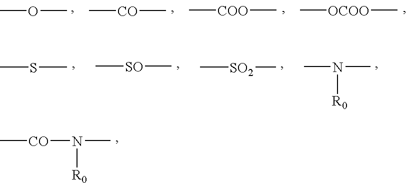

in these formulae, R₀ represents a hydrogen atom, an alkyl radical having from 1 to 4 carbon atoms, a cyclohexyl radical or a phenyl radical.

The compounds of formula (I) preferably employed correspond to the formulae (Ia) and (Ib) in which:

R represents a hydrogen atom, a hydroxyl group, a —CHO group, an —NO₂ group, an —NH₂ group, a linear or branched alkyl or alkoxy radical having from 1 to 6 carbon atoms, preferably from 1 to 4 carbon atoms, or a halogen atom,

B symbolizes a valency bond, an alkylene or alkylidene radical having from 1 to 4 carbon atoms or an oxygen atom,

m is equal to 0 or 1,

n is equal to 0, 1 or 2,

p is equal to 0 or 1.

More preferably still, the choice is made of the compounds of formula (I) in which R represents a hydrogen atom, a hydroxyl group, a methyl radical, a methoxy radical or a halogen atom.

Mention may more particularly be made, by way of illustration of compounds corresponding to the formula (I), of:

halogenated or nonhalogenated aromatic compounds, such as benzene, toluene, chlorobenzene, dichlorobenzenes, trichlorobenzenes, fluorobenzene, difluorobenzenes, chlorofluorobenzenes, chlorotoluenes, fluorotoluenes, bromobenzene, dibromobenzenes, bromofluorobenzenes, bromochlorobenzenes, trifluoromethylbenzene, trifluoromethoxybenzene, trichloromethylbenzene, trichloromethoxybenzene or trifluoromethylthiobenzene,

aminated or nitrated aromatic compounds, such as aniline and nitrobenzene,

phenolic compounds, such as phenol, o-cresol or guaiacol,

monoethers, such as anisole, ethoxybenzene (phenetole), butoxybenzene, isobutoxybenzene, 2-chloroanisole, 3-chloroanisole, 2-bromoanisole, 3-bromoanisole, 2-methylanisole, 3-methylanisole, 2-ethylanisole, 3-ethylanisole, 2-isopropylanisole, 3-isopropylanisole, 2-propylanisole, 3-propylanisole, 2-allylanisole, 2-butylanisole, 3-butylanisole, 2-tert-butylanisole, 3-tert-butylanisole, 2-benzylanisole, 2-cyclohexylanisole, 1-bromo-2-ethoxybenzene, 1-bromo-3-ethoxybenzene, 1-chloro-2-ethoxybenzene, 1-chloro-3-ethoxybenzene, 1-ethoxy-2-ethylbenzene, 1-ethoxy-3-ethylbenzene, 2,3-dimethylanisole or 2,5-dimethylanisole,

diethers, such as veratrole, 1,3-dimethoxybenzene, 1,2-diethoxybenzene, 1,3-diethoxybenzene, 1,2-dipropoxybenzene, 1,3-dipropoxybenzene, 1,2-methylenedioxybenzene or 1,2-ethylenedioxybenzene,

triethers, such as l,2,3-trimethoxybenzene, 1,3,5-trimethoxybenzene or 1,3,5-triethoxybenzene.

The compounds to which the process according to the invention applies in a more particularly advantageous way are benzene, toluene, phenol, anisole and veratrole.

More concisely, the effectiveness of the reactant increases in proportion as the substrate becomes rich in electrons, which, in the case of 6-membered homocyclic nuclei, corresponds to a sum of the Hammett constants σₚ of the possible substituents of less than 0.5 approximately.

The reactant according to the present invention comprises a catalyst according to the present invention, whether a composition or a compound, and an acid anhydride which is preferably an acid halide and generally, for economic reasons, acid chlorides.

In particular, the reactant can comprise a sulfonyl halide of formula (II) R₃SO₂X′. R₃ exhibits an aryl radical, in particular phenyl or naphthyl, optionally substituted by an organic radical, such as a C₁-C₈ alkyl, C₁-C₈ alkyloxy or nitro group, indeed even one or more halogen atoms, in particular chlorine.

R₃ can also be an alkyl radical. X′ represents a halogen atom, preferably a chlorine or bromine atom, or else a residue of another acid in order to form a leaving group. It is simpler to use symmetrical anhydrides or sulfonyl halides.

The reactant can also comprise an acylating reactant, in which case it corresponds to the formula R₃CO—X′ where R₃ and X′ have the same values as above.

In particular, R₃ represents:

a saturated or unsaturated, linear or branched aliphatic radical having from 1 to 24 carbon atoms;

a monocyclic or polycyclic, saturated, unsaturated or aromatic cycloaliphatic radical having from 4 to 12 carbon atoms;

a saturated or unsaturated, linear or branched aliphatic radical carrying a cyclic substituent.

X′ represents:

a halogen atom, preferably a chlorine or bromine atom,

an —O—CO—R₄ radical, with R₄, which is identical to or different from R₃, having the same meaning as R₃.

The term “cyclic substituent” is understood to mean preferably a saturated, unsaturated or aromatic carbocyclic ring, preferably a cycloaliphatic or aromatic ring, in particular a cycloaliphatic ring comprising 6 carbon atoms in the ring or a benzene ring.

More preferably, R₃ represents a linear or branched alkyl radical having from 1 to 12 carbon atoms, preferably from 1 to 6 carbon atoms, it being possible for the hydrocarbonaceous chain optionally to be interrupted by a heteroatom (for example oxygen) or by a functional group (for example —CO—) and/or to carry a substituent (for example a halogen or a CF₃ group).

R₃ preferably represents an alkyl radical having from 1 to 4 carbon atoms, such as methyl, ethyl, propyl, isopropyl, butyl, isobutyl, sec-butyl or tert-butyl.

The R₃ radical also preferably represents a phenyl radical which can optionally be substituted. It is necessary for this radical to be more deactivated than the aromatic compound, because, in the contrary case, the acylating agent itself would be acylated.

Mention may in particular be made, as more specific examples of substituents, of:

a linear or branched alkyl radical having from 1 to 6 carbon atoms, preferably from 1 to 4 carbon atoms, such as methyl, ethyl, propyl, isopropyl, butyl, isobutyl, sec-butyl or tert-butyl,

a linear or branched alkoxy radical having from 1 to 6 carbon atoms, preferably from 1 to 4 carbon atoms, such as the methoxy, ethoxy, propoxy, isopropoxy, butoxy, isobutoxy, sec-butoxy or tert-butoxy radicals,

a hydroxyl group,

a halogen atom, preferably a fluorine, chlorine or bromine atom.

The preferred acylating agents correspond to the formula (II) in which X′ represents a chlorine atom and R₃ represents a methyl or ethyl radical.

When the acylating agent is an acid anhydride, the preferred compounds correspond to the formula (II) in which R₃ and R₄ are identical and represent an alkyl radical having from 1 to 4 carbon atoms.

Mention may more particularly be made, by way of illustration of acylating agents corresponding to the formula (II), of:

acetyl chloride,

monochloroacetyl chloride,

dichloroacetyl chloride,

propanoyl chloride,

isobutanoyl chloride,

pivaloyl chloride,

stearoyl chloride,

crotonyl chloride,

benzoyl chloride,

chlorobenzoyl chlorides,

p-nitrobenzoyl chloride,

methoxybenzoyl chlorides,

naphthoyl chlorides,

acetic anhydride,

isobutyric anhydride,

trifluoroacetic anhydride,

benzoic anhydride.

The reaction can be carried out in a solvent or in the absence of solvent, in which case one of the reactants can be used as reaction solvent, provided that the temperature is at a level where these reactants are molten. A preferred alternative form of the process of the invention consists in carrying out the reaction in an organic solvent.

A solvent for the starting substrate is preferably chosen and more preferably a polar aprotic organic solvent.

Mention may more particularly be made, as examples of polar aprotic organic solvents which can also be employed in the process of the invention, of linear or cyclic carboxamides, such as N,N-dimethylacetamide (DMAC), N,N-diethylacetamide, dimethyl-formamide (DMF), diethylformamide or 1-methyl-2-pyrrolidinone (NMP); nitrated compounds, such as nitromethane, nitroethane, 1-nitropropane, 2-nitropropane or their mixtures, or nitrobenzene; aliphatic or aromatic nitrites, such as acetonitrile, propionitrile, butanenitrile, isobutanenitrile, benzonitrile or benzyl cyanide; dimethyl sulfoxide (DMSO); tetramethyl sulfone (sulfolane), dimethyl sulfone or hexamethylphosphotriamide (HMPT); dimethylethyleneurea, dimethylpropyleneurea or tetramethylurea; or propylene carbonate.

The preferred solvents are: nitromethane, nitroethane, 1-nitropropane or 2-nitropropane.

A mixture of organic solvents can also be used.

Care should be taken, when organic solvents are used, that these solvents, when they are aromatic, are not more nucleophilic than the substrate which it is desired to subject to the reaction.

The amount of catalysts employed is determined so that the ratio of the number of moles of catalysts to the number of moles of acylating or sulfonylating agents or any other agent which can form a cation is less than 1, advantageously than 0.5, preferably than 0.2.

The minimum amount generally corresponds to a ratio at least equal to 0.001, advantageously at least equal to 0.02, preferably to 0.05. The reactions are carried out at atmospheric pressure or under a pressure greater than atmospheric pressure for reasons of simplicity.

The reaction temperature is between 20° C. and 200° C., preferably between 40° C. and 150° C.

Another aspect of the invention relates to a process for preparing the catalyst compound, or promoter, promoter in accordance with the invention.

More specifically, it relates to a process for the preparation of a promoter of formula:

(R₃)_{(μ-q)}Biξ_{q}

comprising at least one ξ⁻ anion, advantageously of formula (I) as defined above,

with:

μ equal to three

ξ corresponding to the formula II

q representing the integer 1 or 2, and

with:

R₃ being as defined above; and

q representing an integer having the value 1 or 2 with, in the case where q is equal to 1, it being possible for the R₃ groups to be identical or different, characterized in that at least one compound of formula (IV):

(R₃)₃Bi (IV)

with R₃ representing

a phenyl group, if appropriate substituted by one or more electron-donating substituents of linear or branched C₁ to C₄ alkyl type, such as, for example, methyl, ethyl or propyl, of C₁ to C₄ alkoxy type, such as methoxy, ethoxy, propoxy or phenoxy, or of C₁ to C₄ thioether type,

a carboxylate group, such as acetate or sulfonate; or

a halogen atom, preferably chlorine, bromine and iodine;

with the R₃ groups being able to be identical or different and preferably being identical,

is reacted with at least one one compound of formula (V):

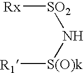

with R₁, R₂ and n being as defined above, and in that said promoter is recovered.

Of course, the stoichiometry between the two components is adjusted according to the degree of deprotometallation desired.

If it is desired to carry out a mono-deprotometallation of the compound of general formula (IV), the compound of general formula (V) is used in a proportion of at most one equivalent.

On the other hand, if it is desired to carry out at least two deprotometallation reactions on the compound of general formula (IV), an excess of compound of general formula (V) is employed.

Furthermore, in the specific case where it is desired to successively carry out three deprotometallation reactions on the compound of general formula (IV), it is advantageous to choose the R₃ groups so as to increase the electron density at the bismuth atom.

This is because the fact that two groups of general formula (I) are already attached to the bismuth atom strongly deactivates the final R₃ group present on this same atom. Consequently, the presence of an electron-rich ligand bonded to the bismuth atom makes it possible to overcome this effect induced by the two groups of general formula (I) and helps in carrying out the final deprotometallation reaction. In this specific case, the R₃ groups present on the bismuth atom are therefore preferably chosen so as to confer, on the latter, a charge at least equivalent to that conferred by three tolyl groups. More preferably, the three R₃ substituents are identical and represent a tolyl group.

The syntheses of the promoters are generally carried out in a solvent of haloalkane type, such as dichloromethane or dichloroethane, or a solvent of acetonitrile type, or toluene, and under an inert atmosphere. The bismuth salt is gradually added to the compound of general formula (V), dissolved beforehand in the cooled solvent.

The expected promoter is subsequently isolated.

This procedure is transposable for the compound according to the invention.

The following nonlimiting examples illustrates the inventions.

### EXAMPLE 1

### Preparation and Isolation of the Mixed Derivative BiCl(OTf)₂

Preparation of BiCl(OTf)₂T

9.11 g (28.89 mmol) of bismuth(III) chloride are introduced into a 100 ml Schenck round-bottomed flask and 60 ml of anhydrous toluene are added. 10.5 g (70 mmol) of triflic acid are then added under cold conditions. The suspension is stirred magnetically, the round-bottomed flask is connected to an oil bubbler and is heated at 110° C. using an oil bath for 1 h 30. At the end of this time, no more evolution of HCl is observed in the bubbler. The mixture is cooled and the toluene is removed using a syringe. The white paste is washed with 50 ml of anhydrous dichloromethane. After evaporating the solvents under vacuum (0.1 mmHg) and by heating at 60° C., 14.04 g of a white powder with a pearlescent appearance are recovered, i.e. an isolated yield of 89%.

Spectroscopic Characteristics:

¹⁹F NMR (δ in CD₃CN): 0.94;

¹³C NMR (δ in d6-DMSO):125.9 (J=322 Hz);

IR analysis (cm⁻): 1326(m), 1271(m), 1232(m), 1201(s), 1032(m), 1022(m), 1001(m)

Raman analysis: 1303, 1293, 1250, 1213, 1175, 1154, 1054, 781, 654, 584, 518, 365, 351, 337, 308.

### EXAMPLE 2

### SbCl(OTf)₂—Preparation of SbCl(OTf)₂

5 g (21.92 mmol) of antimony(III) chloride are introduced into a 100 ml Schenck round-bottomed flask and 60 ml of anhydrous toluene are added. 7.24 g (48.22 mmol) of triflic acid are then added under cold conditions. The solution is stirred magnetically, connected to an oil bubbler and heated at 110° C. using an oil bath for 5 h. At the end of this time, no more evolution of HCl is observed in the bubbler. The mixture is cooled and the toluene is removed using a syringe. The white paste is washed under cold conditions (ice bath) with 2×50 ml of anhydrous dichloromethane. After evaporating the solvents under vacuum (0.1 mmHg) and by heating at 60° C., 4.6 g of a white powder with a pearlescent appearance are recovered, i.e. an isolated yield of 46%.

Spectroscopic Characteristics:

¹⁹F NMR (δ in d6-DMSO): 1.51

¹³C NMR (δ in d6-DMSO): 120.5 (J=322 Hz);

Raman analysis: 1330, 1315, 1230, 1134, 1017, 774, 646, 589, 517, 376, 360, 356, 345, 331, 253, 166

### EXAMPLE 3

### Catalytic Systems Tested for Alkanesulfonylation

Procedure

The aromatic compound tested is brought into contact with mesyl chloride in an equimolar ratio of 1. The catalyst is then introduced and the reaction is then carried out for 24 h at a temperature of 105° C. The catalyst is introduced in a proportion of 10 mol % with respect to the amount of substrate introduced. The results are collated in the table below,

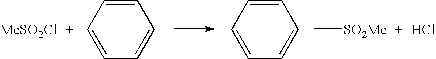

| | Molar | | | |
|---|---|---|---|---|
| | proportion of | | | |
| | catalyst with | | | |
| Catalytic | respect to the | | Operating | |
| System | substrate | Yield | condition | Observation |
| BiCl₃ | 10% | 0 | −24 hours | comparative |
| TfOH | 10% | 0 | −24 hours | comparative |
| SbCl₃ | 10% | 60% | 24 hours | |

### EXAMPLE 4

### Methanesulfonylation of Other ArH Compounds

ArH gives ArSO₂Me

Molar proportion of catalyst with respect to the substrate 10%

| Catalytic | | | | |
|---|---|---|---|---|
| System | Substrate | Yield | Operating conditions | Observations |
| TfOH + SbCl₃ | Fluoro | 97% | 105° C. three | |
| | benzene | | days | |
| TfOH + GaCl₃ | Benzene | 97% | 105° C. eight | |
| | | | hours | |
| TfOH + SbCl₃ | Benzene | 31% | 105° C. eight | |
| | | | hours | |

The combinations of the triflic with the following metal chlorides have also been tested positively: antimony(III) chloride, antimony(V) chloride, tin(IV) chloride and tin(IV) chloride pentahydrate.

Furthermore, the combinations of the triflic with bismuth oxychloride and bismuth oxide are active.

Finally, the use of triflic monohydrate also results in active systems, which has been demonstrated in the case of the system with gallium chloride. From the latter position, it could be inferred therefrom that the use of a vigorously anhydrous medium is not necessary.

### EXAMPLE 5

### BiPh₂(NTf₂)

Tf₂NH (0.281 g; 1 mmol) is introduced into 10 ml of distilled CH₂Cl₂ in a 100 ml Schlenck flask purged with argon. The Schlenck flask is cooled to 0° C. A solution of BiPH₃ (0.44 g: 1 mmol) in 10 ml of CH₂Cl₂ is added with a syringe. The mixture assumes an orangey yellow color and a compound insoluble in dichloromethane appears. The Schlenck flask is brought back to [lacuna] temperature and stirring is maintained for three hours. All the dichloromethane is evaporated off and the residue is dried under vacuum. A white BiPh₂(NTf₂) powder is obtained (0.60 g, 0.94 mmol, Yd 94%).

Spectroscopic Characteristics of BiPh₂(NTf₂):

¹H NMR (400, 13 MHz): δ: 7.50 (para, 1H, Hₓ, tt, J(HₓHₘ)=7.5 Hz, J(HₓHₐ)=1.2 Hz), 7.89 (meta, 2H, Hₘ, dd, J(HₘHₓ)=7.5 Hz, J(HₘHₐ)=7.8 Hz), 8.52 (ortho, 2H, Hₐ, dd, J(HₐHₘ)=7.8 Hz, J(HₐHₓ)=1.2 Hz).

¹⁹F NMR (376.48 MHz): singlet at δ=−1.79 ppm.

¹³C NMR (100.62 MHz): δ: 121.0 (q, J=321 Hz, CF₃), 131.3 (s, CH), 133.7 (s, CH), 186.6 (s, CH), ipso Cq of the aromatic ring not displayed by NMR.

### EXAMPLE 6

### BiPh(NTf₂)₂

This is the same process as that described for BiPh₂(NTf₂), starting from 2 mmol of Tf₂NH and 1 mmol of BiPH₃. A white BiPh(NTf₂)₂ powder is obtained (0.76 g, 0.9 mmol, 90%).

Spectroscopic Characteristics of BiPh(NTf₂)₂:

¹H NMR (400.13 Mz): δ: 7.60 (para, 1H, Hₓ, tt, J(HₓHₘ)=7.5 Hz, J(HₓHₐ)=1.2 Hz), 8.32 (meta, 2H, Hₘ, dd, J(HₘHₓ)=7.5 Hz, J(HₘHₐ)=8.3 Hz), 9.21 (ortho, 2H, Hₐ, dd, J (HₐHₘ)=8.3 Hz, J (HₐHₓ)=1.2 Hz)

¹⁹F NMR (75.393 MHz): singlet at δ=−2.1 ppm.

¹³C NMR (75.469 MHz): δ: 120.5 (q, J=321 Hz, CF₃), 130.7 (s, CH), 135.1 (s, CH), 138.8 (s, CH), ipso Cq of the aromatic ring not displayed by NMR.

### EXAMPLE 7

### Bi(NTf₂)₃

A solution of Tf₂NH (0.85 g, 3 mmol) in 10 ml of CH₂Cl₂ is introduced under argon into a 100 ml Schlenck flask. The Schlenck flask is cooled in an ice bath and a solution of Bi(Tolyl)₃ (0.48 g, 1 mmol) in 10 ml of CH₂Cl₂ is added with a syringe. The mixture instantaneously assumes an orangey yellow color and an insoluble compound appears. After stirring overnight at ambient temperature, the solvents are evaporated under vacuum. 1.01 g of a pale yellow Bi(NTf₂)₃ powder are thus recovered, i.e. a yield of 96%. This product is stored and handled in a glove box.

Spectroscopic Characteristics of Bi(NTf₂)₃:

¹H NMR (300.13 MHz): Absence of peaks

¹⁹F NMR (376.47 MHz): singlet at δ=−1.77 ppm.

¹³C NMR (75.469 MHz): δ: 120.4 (q, J=321 Hz, CF₃).

IR (CCl₄) ν (cm⁻¹): 1451 (very strong), 1305 (shoulder), 1231 (very strong), 1132 (very strong), 894 (shoulder), 855 (very strong), 650 (strong), 608 (very strong), 573 (shoulder), 502 (very strong).

### EXAMPLE 8

### Catalytic Benzoylation of Toluene

All the handling is carried out under argon. Toluene (4.6 g, 50 mmol), tetradecane (0.496 g, 2.5 mmol) and 5 mmol of the chosen acylating agent (benzoic anhydride or benzoyl chloride) are successively introduced into a 50 ml two-necked flask equipped with a reflux condenser and containing beforehand Bi(NTf₂)₃ (0.525 g, 500 μmol). The reaction mixture, with stirring, is placed in an oil bath at 110° C. The progress of the reaction is monitored by GC by withdrawing, with a syringe, a small portion of the reaction mixture in order to determine the change in the methylbenzophenone (ortho, meta and para) yield. This analysis is complemented by comparison of the chromatogram and mass spectra (GC/MS) obtained with pure samples of o-, m- and p-methylbenzophenone [Aldrich, 15,753-8, 19,805-6 and M2,955-9]. Percentage of ortho/meta/para isomers: 16/4/80 (from benzoyl chloride), 20/4/76 (from benzoic anhydride).

GC: Analytical condition: Starting temperature=125° C.

Final temperature=300° C.

Slope=20° C./min

Retention times: ortho: 6.1 min; meta: 6.4 min; para: 6.6 min.

GC/MS [m/z (%)]:

o-methylbenzophenone: 196 (M⁺, 60), 195(100), 119(24), 105(55), 91(41), 77(89).

p-methylbenzophenone: 196 (M⁺, 57), 181(12), 119(100), 105(43), 91(41), 77(61).

After 4 hours, a cumulative yield of various isomers of 60% is obtained.

The monophenylated derivative BiPh(NTf₂)₂ gives a 55% yield.

### EXAMPLE 9

### Catalytic Sulfonylation of Toluene

This is the same process as that described for the benzoylation. This analysis is also complemented by comparison of the chromatogram and mass spectra (GC/MS) obtained with pure samples of o-, m- and p-methyldiphenyl sufone.

GC: Analytical condition: Starting temperature=125° C.

Final temperature=300° C.

Slope=20° C./min

Retention times: ortho: 7.8 min; meta: 7.9 min; para: 8.1 min.

Percentage of ortho/meta/para isomers: 34/6/60 (from benzenesulfonyl chloride).

GC/MS [m/z (%)]:

o-methyldiphenyl sulfone: 232 (M⁺, 25), 214(45), 166(72), 137(33), 91(35), 77(100),

p-methyldiphenyl sulfone: 232 (M⁺, 65), 139(75), 125(52), 107(67), 91(48), 77(100).

The derivative obtained by the action of of excess triflimide on bismuth trichloride (inferred formula BiCl₂(NTf₂)) gives, after 5 h, about the same yield as Bi(NTf₂)₃ for an identical amount of bismuth, namely approximately 35%.

Under these conditions, neither bismuth chloride nor triflic leads to sulfonylation.

### EXAMPLE 10

### Activation of the Carbonyl by a Lewis Acid and Addition to an Unsaturation, Such as Enol

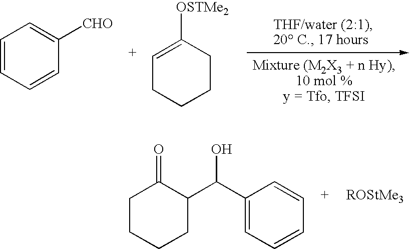

General Procedure

* Aldolization reaction

Reactions with Isolated Rare Earth Metal Triflates or Triflimides

The rare earth metal triflate (TfO⁻) or triflimide (TfSI⁻) (0.04 mmol) is diluted in a THF/water (2 ml/1 ml) mixture at ambient temperature in a 40 ml Schott tube. Benzaldehyde (0.4 mmol) and silylated enol ether (0.4 mmol) are successively added to this solution. The mixture is stirred at 20° C. for 17 h and then analyzed by LC with external calibration.

Reactions with “Preparing” Solutions of Isolated Rare Earth Metal Triflates or Triflimides

The rare earth metal source (2 mmol) is suspended in water (2 ml) in a 40 ml Schott tube. Triflic acid or triflimide (n*2 mmol) is added at ambient temperature and the reaction medium is brought to reflux for 3 h. After returning to 20° C., this solution is used in the aldolization reaction instead of the isolated rare earth metal triflate or triflimide (see above procedure).

The results are collated in the table of example 5.

The difference between the results of the isolated triflates and of the triflates prepared in situ, with n=6, is attributed to the presence of mixed salts according to the invention.

### EXAMPLE 11

### Acylation

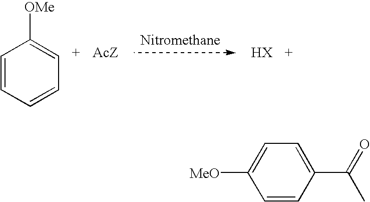

General Procedure

* Acylation reaction

The acylating agent (10 mmol) and then lanthanum triflate or triflimide or the equivalent in preparing solution are added at 20° C. to a solution of anisole (5 mmol) in nitromethane (5 ml) in a 25 ml round-bottomed flask equipped with a magnetic bar stirrer. The reaction medium is heated at 50° C. for 4 h and then analyzed by GC.

The difference between the results of the isolated triflates and of the triflates prepared in situ, with n=6, is attributed to the presence of mixed salts according to the invention.

Table of Example 5

| [Illegible] | [Illegible] | [Illegible] | [Illegible] | [Illegible] | [Illegible] | [Illegible] |
|---|---|---|---|---|---|---|
| Yb₂O₃ | TfOH | 6 | 79.5 | 81 | 98 | 77 |
| Yb₂O₃ | TfOH | 4 | 82 | 83 | 98 | 77 |
| Yb₂O₃ | TfOH + TFSIH^{(d)} | 6 | 75 | 77 | 97 | 77 |
| Nd₂(CO₃)₃ | TFSIH | 4 | 32 | 35 | 91 | 64 |
| Nd₂(CO₃)₃ | TfOH + TFSIH^{(d)} | 6 | 29 | 33 | 88 | 64 |
| La₂O₃ | TfOH | 4 | 20 | 21 | 95 | 19.5 |
| La₂(CO₃)₃ | TfOH | 6 | 53 | 55 | 96 | 19.5 |
| La₂(CO₃)₃ | TfOH | 4 | 45 | 48 | 94 | 19.5 |
| La₂(PO₄)₃ | TfOH | 4 | 41 | 46 | 89 | 19.5 |

^{(a)}Quantitative determination by LC with external calibration, expressed in mol %
^{(b)}DC of the PhCHO. expressed in mol %
^{(c)}CY = RY/DC, expressed in mol %
^{(d)}TfOH + TFSIH as a 1:1 mol to mol mixture.

TABLE OF EXAMPLE 6

| | | | | | | |
|---|---|---|---|---|---|---|
| [Illegible] | [Illegible] | [Illegible] | [Illegible] | [Illegible] | [Illegible] | [Illegible] |

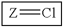

| | | | | | | |
|---|---|---|---|---|---|---|
| La₂(CO₃)₃ | TfOH | 6 | 64 | 84 | 78 | 44 |
| La₂(CO₃)₃ | TfOH | 4 | 61 | 84 | 73 | 44 |
| La₂(CO₃)₃ | TFSIH | 6 | 53 | 85 | 81 | 44 |
| La₂(CO₃)₃ | TFSIH | 4 | 50 | 70 | 71 | 44 |

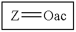

| | | | | | | |
|---|---|---|---|---|---|---|
| La₂(CO₃)₃ | TfOH | 6 | 65 | 83 | 78 | 44 |
| La₂(CO₃)₃ | TfOH | 4 | 62 | 81 | 76 | 44 |
| La₂(CO₃)₃ | TFSIH | 6 | 59 | 68 | 87 | 44 |
| La₂(CO₃)₃ | TFSIH | 4 | 55 | 66 | 83 | 44 |

(a) Quantitative determination by GC with internal calibration, expressed in mol %
(b) DC of the anisole, expressed in mol %
(c) CY = RY/DC, expressed in mol %.

1. Use, as catalyst, of salts of elements of valency μ, with μ at least equal to 3, comprising, as coanions, at least 1 and at most (μ−1) anions carrying a sulfonyl functional group carried by a perhalogenated atom, preferably a perfluorinated atom, more preferably a perfluoromethylene (—CF₂—) group.

2. Use according to claim 1, characterized in that said salt corresponds to the formula:
MY_{μ-q}ξ_{q}
where M represents a μ-valent and at least trivalent element in the cationic form, preferably known to give lewis acids;
where Y is a monovalent anion or a monovalent anionic functional group and
where ξ⁻ represents an anion or an anionic functional group carrying a sulfonyl functional group carried by a perhalogenated atom, preferably a perfluorinated atom, more preferably a perfluoromethylene (—CF₂—) group and
where q is an integer advantageously chosen within the closed range (comprising the limits) ranging from 1 to (μ−1) (that is to say, 1 or 2 when μ is 3).

3. Use according to claims 1 and 1, characterized in that said: ξ⁻ corresponds to the formula:
—R₁—Z—SO₂—Rₓ
where Z represents an atom from the nitrogen column or a chalcogen;
where, when Z represents an atom from the nitrogen column, R₁ represents an electron-withdrawing radical;
where Rₓ is a radical in which the atom, generally a carbon atom, carrying the sulfonyl functional group is perhalogenated, advantageously Rₓ is R_{f} of formula:
EWG—(CX₂)ₚ—
in which:
the X groups, which are alike or different, represent a fluorine or a radical of formula CₙF₂ₙ₊₁, with n an integer at most equal to 5, preferably to 2;
p represents zero or an integer at most equal to 2, with the proviso that, when p represents zero, EWG is chlorine and especially fluorine;
EWG represents a hydrocarbonaceous group, advantageously an electron-withdrawing group (that is to say, the Hammett constant σₚ of which is greater than 0, advantageously than 0.1, preferably than 0.2), the possible functional groups of which are inert under the reaction conditions, preferably fluorine or a perfluorinated residue of formula CₙF₂ₙ₊₁, with n an integer at most equal to 8, advantageously to 5.

4. Use according to claims 1 to 3 of salts of elements of valency μ, with μ at least equal to 3, comprising, as coanions, at least 1 and at most (μ−1) sulfonate anions in which the sulfonic functional group is carried by a perhalogenated atom, preferably a perfluorinated atom, more preferably a perfluoromethylene (—CF₂—) group.

5. Use according to claim 4, characterized in that said use is the use as catalyst of Lewis acid type.

6. Use according to claims 1 to 5, characterized in that said salt corresponds to the formula:
MY_{3-q}[(Rₓ)—SO₂—O⁻]_{q}
with M represents an at least trivalent element in the cationic form, preferably known for giving Lewis acids, where Y is a monovalent anion or a monovalent anionic functional group and where Rₓ is a radical in which the carbon carrying the sulfonic functional group is perhalogenated and where q is an integer advantageously chosen between 1 and 2 (that is to say, 1 or 2).

7. Use according to claims 1 to 6, characterized in that said salt is a salt of formula:
MY_{μ-q}(RₓSO₂—O⁻)_{q},
where M is an element in an at least trivalent cationic form;
where μ represents the charge of the cation corresponding to M;
where Y represents the anion or anions, other than the sulfonates perhalogenated on the carbon carrying said sulfonate functional group;
where q represents an integer chosen within the closed range from 1 to μ−1.

8. Use according to claims 1 to 7, characterized in that said element is chosen from rare earth metals (scandium, yttrium, lanthanum and lanthanide) and elements forming a square in the Periodic Table composed of gallium, germanium, arsenic, indium, tin, antimony, thallium, lead and bismuth.

9. Use according to claims 1 to 8, characterized in that said salt is a trivalent metal salt comprising, as coanions, at least 1 and at most 2 sulfonate anions in which the sulfonic functional group is carried by a perhalogenated atom, preferably a perfluorinated atom, more preferably a perfluoromethylene (—CF₂—) group.

10. Use according to claims 1 to 9, characterized in that said salt corresponds to the formula:
MY_{3-q}[(Rₓ)—SO₂—O⁻]_{q}
with M representing a trivalent metal, preferably known for giving Lewis acids, where Y is a monovalent anion or a monovalent anionic functional group and where Rₓ is a radical in which the carbon carrying the sulfonic functional group is perhalogenated and where q is an integer advantageously chosen between 1 and 2 (that is to say, 1 or 2).

11. Catalytic composition, characterized in that it comprises one or more compounds corresponding to the empirical formula:
MY_{3-q}[(Rₓ)—SO₂—O⁻]_{q}
with M representing an at least trivalent element, preferably known for giving Lewis acids, where Y is a monovalent anion or a monovalent anionic functional group and where Rₓ is a radical in which the carbon carrying the sulfonic functional group is perhalogenated and where q is between 0.1 and 2.9, advantageously from 0.5 to 2.5, preferably from 1 to 2, inclusive.

12. Catalytic composition according to claim 11, characterized in that it is obtained, advantageously in situ, by introduction of at least one acid ξH onto a salt MY_{μ} where M is advantageously chosen from [lacuna] earth metals, gallium, germanium, arsenic, indium, tin, antimony, thallium and lead.

13. Compound of formula:
MY_{μ-q}(RₓSO₂—O⁻)_{q},
where M is an element in an at least trivalent cationic form;
where μ represents the charge of the cation corresponding to M;
where Y represents the anion or anions, other than the sulfonates perhalogenated on the carbon carrying said sulfonate functional group;
where q represents an integer chosen within the closed range from 1 to μ−1.

14. Compound according to claim 13 of formula:
MY_{3-q}[(Rₓ)—SO₂—O⁻]_{q}
with M representing a trivalent metal, preferably known for giving Lewis acids, where Y is a monovalent anion or a monovalent anionic functional group and where Rₓ is a radical in which the carbon carrying the sulfonic functional group is perhalogenated and where q is an integer chosen between 1 and 2 (that is to say, 1 or 2).

15. Reactant comprising:
a catalytic composition according to claim 11;
an agent capable of giving carbocations in the presence of Lewis acid chosen from acid anhydrides, in particular carboxylic and sulfonic anhydrides, carbonyls, in particular aldehydes, or conjugated dienes.

16. Reactant comprising:
a catalytic composition according to claim 11;
an oxygen-comprising heterocycle, chosen in particular from cyclic ethers and lactones.
